(19) United States
(12) Patent Application Publication
(10) Pub. No.: US 2023/0293228 A1
(43) Pub. Date: Sep. 21, 2023

(54) Expandable multilayered electrode elements for thrombectomy procedures

(71) Applicant: MAGNETO THROMBECTOMY SOLUTIONS LTD., Or Yehuda (IL)

(72) Inventors: Yuval Taff, Tel Aviv (IL); Assaf Torjman, Herzliya (IL)

(21) Appl. No.: 18/020,435

(22) PCT Filed: Sep. 14, 2021
(86) PCT No.: PCT/IB2021/058330

§ 371 Date: Feb. 9, 2023

Related U.S. Application Data
(60) Provisional application No. 63/078,920, filed on Sep. 16, 2020.

(51) Int. Cl. A61B 18/14

(52) U.S. Cl. CPC A61B 18/1492; A61B 2018/0041

(57) ABSTRACT

An apparatus (20) includes one or more longitudinal elements (28a, 28b, 30, 60) configured to pass through a sheath (22) within a body of a subject, and one or more expandable multilayered electrode elements (24) coupled to the longitudinal elements. The electrode elements are configured to advance to a thrombus in the body while collapsed inside the sheath and to expand distally to the sheath following the advance to the thrombus. Each of the electrode elements includes one or more reference electrodes (34) and one or more active electrodes (36) configured to attract the thrombus, following the expansion of the electrode elements, upon application of a voltage between the active electrodes and the reference electrodes. Other embodiments are also described.

### CROSS-REFERENCE TO RELATED APPLICATIONS

The present application claims the benefit of U.S. Provisional Application 63/078,920, entitled “Expandable electrode elements for thrombectomy procedures,” filed Sep. 16, 2020, whose disclosure is incorporated herein by reference.

### FIELD OF THE INVENTION

The present invention relates to the field of medical devices, particularly devices for thrombectomy procedures.

### BACKGROUND

U.S. Pat. No. 10,028,782 to Orion, whose disclosure is incorporated herein by reference, describes a flexible catheter device capable of being introduced into a body passage and withdrawing fluids therefrom or introducing fluids thereinto. The device includes electrodes configured to apply electrical signals in the body passage for carrying out thrombus dissolution and/or thrombectomy, wherein one of said electrodes is designed to contact the thrombus material and remove it or dissolve it, and wherein the electrical voltage signals are unipolar pulsatile voltage signals.

US Patent Application Publication 2018/0116717 to Taff et al., whose disclosure is incorporated herein by reference, describes an apparatus for removal of a thrombus from a body of a subject. The apparatus includes a first electrode, made of a first conductive metal, a second electrode, made of a second conductive metal that is different from the first conductive metal, and a voltage source, configured to apply a positive unipolar voltage between the first electrode and the second electrode while the first electrode is in contact with the thrombus, and while the second electrode is inside the body of the subject.

US Patent Application Publication 2019/0262069 to Taff et al., whose disclosure is incorporated herein by reference, describes an apparatus that includes an electrically-insulating tube, which includes a distal end having a circumferential wall that is shaped to define one or more perforations, configured for insertion into a body of a subject, an outer electrode, disposed over the distal end of the electrically-insulating tube, and configured to lie at least partly within a thrombus while the electrically-insulating tube is inside the body, and an inner electrode, configured to lie, within the tube, opposite the perforations, while the outer electrode lies at least partly within the thrombus. The outer electrode is configured to attract the thrombus while the outer electrode lies at least partly within the thrombus and the inner electrode lies opposite the perforations, when a positive voltage is applied between the outer electrode and the inner electrode such that electric current flows through the perforations.

US Patent Application Publication 2021/0186540 to Taff et al., whose disclosure is incorporated herein by reference, describes an apparatus including a tube. The tube is configured to advance to a blockage and includes a proximal hub configured to connect to a suction-applying device such that, following the advancement of the tube to the blockage, a suction force generated by the suction-applying device is applied, via the tube, to the blockage. The apparatus further includes a control element, including first and second electrically-conductive circumferential portions, configured to pass through the tube. The apparatus further includes first and second electrically-conductive elements, configured to connect the first and second electrically-conductive circumferential portions to respective terminals of a power source. The first electrically-conductive circumferential portion is configured to attract the blockage when a voltage is applied by the power source, via the first and second electrically-conductive elements, between the first and second electrically-conductive circumferential portions, such that the blockage is anchored to the control element while the suction force is applied to the blockage.

International Patent Application Publication WO/2019/243992 to Taff et al., whose disclosure is incorporated herein by reference, describes an apparatus for removing a blockage from a body of a subject. The apparatus includes a reference electrode, configured for insertion into the body, an electrically-insulative element covering the reference electrode, the electrically-insulative element being shaped to define a gap that exposes a portion of the reference electrode, an active electrode covering the electrically-insulative element, and an electrically-conductive element passing through the reference electrode and electrically connected to the active electrode, the electrically-conductive element being configured to electrically connect the active electrode to a power source such that application, by the power source, of a voltage between the active electrode and the reference electrode causes the active electrode to attract the blockage.

International Patent Application Publication WO/2020/174326 to Taff et al., whose disclosure is incorporated herein by reference, describes an apparatus for treating a blockage in a body of a subject, including a tube configured for insertion into the body and shaped to define: a first lumen, and a second lumen having a distal opening. The apparatus further includes a pair of electrodes configured to apply an electric current to the blockage upon application of a voltage between the electrodes, the pair including an outer electrode wrapped around the tube and an inner electrode configured to pass through the first lumen.

### SUMMARY OF THE INVENTION

There is provided, in accordance with some embodiments of the present invention, an apparatus including one or more longitudinal elements, configured to pass through a sheath within a body of a subject, and one or more expandable multilayered electrode elements coupled to the longitudinal elements. The electrode elements are configured to advance to a thrombus in the body while collapsed inside the sheath, and to expand distally to the sheath following the advance to the thrombus. Each of the electrode elements includes one or more reference electrodes and one or more active electrodes configured to attract the thrombus, following the expansion of the electrode elements, upon application of a voltage between the active electrodes and the reference electrodes.

In some embodiments, the apparatus further includes the sheath.

In some embodiments, the longitudinal elements include a proximally-coupled longitudinal element coupled to respective proximal ends of the electrode elements.

In some embodiments, the longitudinal elements include a distally-coupled longitudinal element coupled to respective distal ends of the electrode elements.

In some embodiments, the distally-coupled longitudinal element includes:
a longitudinal-element active electrode;
a longitudinal-element reference electrode disposed inside the longitudinal-element active electrode; and
an electrically-insulating element disposed between the longitudinal-element reference electrode and the longitudinal-element active electrode.

In some embodiments, the distally-coupled longitudinal element extends distally to the electrode elements.

In some embodiments, the electrode elements are configured to expand so as to define one or more loops.

In some embodiments, the apparatus further includes a distal electrode element disposed at respective distal ends of the loops and including:
a distal active electrode;
a distal reference electrode disposed inside the distal active electrode; and
a distal electrically-insulating element disposed between the distal reference electrode and the distal active electrode.

In some embodiments, the loops include:
a proximal set of one or more proximal loops; and
a distal set of one or more distal loops coupled to respective distal ends of the proximal loops, the distal set having a maximal width that is less than that of the proximal set.

In some embodiments, at least one of the electrode elements has a sinusoidal shape when expanded.

In some embodiments, at least one of the electrode elements has a helical shape when expanded.

In some embodiments, the electrode elements are configured to expand to define a shape having a width that decreases moving distally along a distal portion of the shape.

In some embodiments, each of the electrode elements includes a multilayered strip.

In some embodiments,
a reference layer of the strip includes the reference electrodes,
one or more active layers of the strip include the active electrodes, and
the strip further includes one or more insulating layers that electrically insulate the reference layer from the active layers.

In some embodiments, the active layers consist of a single active layer, and the insulating layers consist of a single insulating layer disposed between the reference layer and the active layer.

In some embodiments,
the active layers include a first active layer and a second active layer disposed on opposite sides of the reference layer, and
the insulating layers include:
   a first insulating layer disposed between the first active layer and the reference layer; and
   a second insulating layer disposed between the second active layer and the reference layer.

In some embodiments, each of the active layers is shaped to define one or more outer gaps, and each of the insulating layers is shaped to define one or more inner gaps aligned with the outer gaps.

In some embodiments, at least one of the insulating layers is narrower than (i) the reference layer, or (ii) an adjacent one of the active layers.

In some embodiments, the strip includes:
a substrate layer; and
one or more electrode layers mounted to the substrate layer, each of the electrode layers including a respective one of the reference electrodes and a respective one of the active electrodes.

In some embodiments, the respective one of the reference electrodes and the respective one of the active electrodes protrude into one another.

In some embodiments, each of the electrode elements includes:
a core, which includes the reference electrodes;
an electrically-insulating layer, which is wrapped around the core; and
an active layer, which includes the active electrodes and is wrapped around the electrically-insulating layer.

In some embodiments, the active layer is shaped to define one or more outer gaps, and the electrically-insulating layer is shaped to define one or more inner gaps at least partly aligned with the outer gaps.

There is further provided, in accordance with some embodiments of the present invention, an apparatus including one or more expandable electrode elements configured to advance to a thrombus in a body of a subject while collapsed inside a sheath within the body, and to expand distally to the sheath so as to define one or more loops following the advance to the thrombus. The apparatus further includes an electrically-conductive longitudinal element coupled to respective distal ends of the electrode elements and configured to pass through the sheath. Each of the electrode elements includes one or more active electrodes configured to attract the thrombus, following the expansion of the electrode elements, upon application of a voltage between the active electrodes and the longitudinal element.

There is further provided, in accordance with some embodiments of the present invention, a method including inserting a sheath into a body of a subject, advancing one or more expandable multilayered electrode elements to a thrombus in the body while the electrode elements are collapsed inside the sheath, each of the electrode elements including one or more reference electrodes and one or more active electrodes, causing the electrode elements to expand distally to the sheath following the advance to the thrombus, and causing the active electrodes to attract the thrombus, following the expansion of the electrode elements, by applying a voltage between the active electrodes and the reference electrodes.

In some embodiments, the method further includes, following the expansion of the electrode elements, twisting the electrode elements around the thrombus by rotating one or more longitudinal elements that are coupled to the electrode elements and pass through the sheath.

In some embodiments,
a longitudinal element is coupled to respective distal ends of the electrode elements and includes:
   a longitudinal-element active electrode, and
   a longitudinal-element reference electrode disposed inside the longitudinal-element active electrode, and
the method further includes applying the voltage between the longitudinal-element active electrode and the longitudinal-element reference electrode.

In some embodiments,
a distal electrode element is disposed at respective distal ends of the loops and includes:
   a distal active electrode, and
   a distal reference electrode disposed inside the distal active electrode, and
the method further includes applying the voltage between the distal active electrode and the distal reference electrode.

There is further provided, in accordance with some embodiments of the present invention, a method including inserting a sheath into a body of a subject. The method further includes advancing one or more expandable electrode elements to a thrombus in the body while the expandable electrode elements are collapsed inside the sheath, each of the electrode elements including one or more active electrodes, and an electrically-conductive longitudinal element being coupled to respective distal ends of the electrode elements. The method further includes causing the electrode elements to expand distally to the sheath so as to define one or more loops following the advance to the thrombus, and causing the active electrodes to attract the thrombus, following the expansion of the electrode elements, by applying a voltage between the active electrodes and the longitudinal element.

The present invention will be more fully understood from the following detailed description of embodiments thereof, taken together with the drawings, in which:

### BRIEF DESCRIPTION OF THE DRAWINGS

FIG. 1 is a schematic illustration of an apparatus for removal of a thrombus from a body of a subject, in accordance with some embodiments of the present invention;

FIGS. 2A-D show transverse cross-sections through an electrode element, in accordance with different respective embodiments of the present invention;

FIG. 2E is a schematic illustration of an electrode element comprising a multilayered strip, in accordance with some embodiments of the present invention;

FIG. 2F is a schematic illustration of a transverse cross-section through an electrode element, in accordance with some embodiments of the present invention; and

FIGS. 3-5 are schematic illustrations of an apparatus for removal of a thrombus from a body of a subject, in accordance with different respective embodiments of the present invention.

### DETAILED DESCRIPTION OF EMBODIMENTS

### Overview

Per some thrombectomy techniques, a positive voltage is applied between an active electrode, which contacts or is at least adjacent to a thrombus within the body of a subject, and a reference electrode. The applied voltage causes the negatively-charged thrombus to adhere to the positively-charged active electrode. Following the adhesion of the thrombus to the active electrode, the electrodes, together with the thrombus, are withdrawn from the body.

A challenge, when performing these techniques, is that the electric current resulting from the applied voltage, and hence the force of attraction between the active electrode and the thrombus, may be distributed relatively unevenly over the length of the thrombus.

To address this challenge, embodiments of the present invention provide an apparatus comprising an expandable multilayered electrode element. One layer of the electrode element functions as a reference electrode, while one or more other layers function as active electrodes. The electrode element is expanded so as to pass through and/or surround the thrombus along most or all of the length of the thrombus. Subsequently, a voltage is applied between the active electrodes and the reference electrode. By virtue of the electrodes extending across most or all of the length of the thrombus, the resulting electric current is distributed relatively evenly over the length of the thrombus.

In some embodiments, the expandable electrode element comprises a multilayered strip. For example, the expandable electrode element may comprise three layers: an active-electrode layer, a reference-electrode layer, and a middle insulating layer between the active-electrode layer and reference-electrode layer. Alternatively, the expandable electrode element may comprise five layers, including two active-electrode layers and two insulating layers.

Optionally, additional features of the multilayered strip may facilitate a greater flow of current between the active-electrode layer(s) and reference-electrode layer. Such features may include gaps in, and/or a decreased width of, the insulating layer(s).

In other embodiments, the expandable electrode element comprises a reference-electrode core, an electrically-insulating layer wrapped around the core, and an active-electrode layer wrapped around the electrically-insulating layer. Gaps in the electrically-insulating layer may facilitate the flow of current between the active-electrode layer and the reference-electrode core.

In some embodiments, the apparatus comprises one or more electrode elements configured to expand so as to define one or more loops, which may entrap the thrombus. The distal ends of the loops may be coupled to a longitudinal element passing through the sheath, which, in addition to facilitating control over the electrode elements, may function as an additional electrode element. Alternatively or additionally, a distal electrode element may be disposed at the distal ends of the loops.

In other embodiments, the electrode elements expand so as to define another shape, such as a sinusoidal or helical shape.

### Apparatus Description

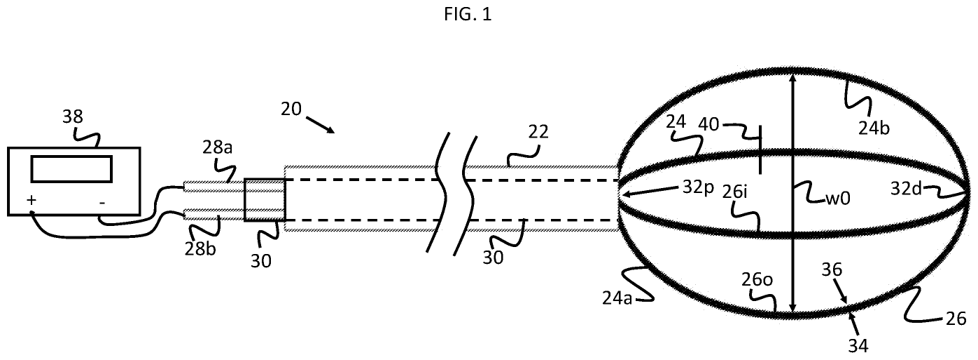

Reference is initially made to FIG. 1, which is a schematic illustration of an apparatus 20 for removal of a thrombus from a body of a subject, in accordance with some embodiments of the present invention.

Apparatus 20 comprises a sheath 22 configured for insertion into the body, typically via a femoral, jugular, carotid, or radial vein of the subject. Subsequently to the insertion of sheath 22, the sheath is navigated to the thrombus, which is typically located inside a blood vessel of the subject. For example, the thrombus may be located inside a pulmonary, carotid, femoral, popliteal, tibial, or peroneal artery or vein of the subject.

In some embodiments, sheath 22 is radiopaque, and the sheath is navigated under fluoroscopy. Alternatively or additionally, the sheath may be navigated over a guidewire and/or through a delivery catheter.

Typically, the sheath comprises a flexible polymer. In some embodiments, the length of the sheath is between 30 and 150 cm.

Apparatus 20 further comprises one or more expandable multilayered electrode elements 24 configured to advance to the thrombus while collapsed inside sheath 22. For example, following the navigation of sheath 22 to the thrombus, electrode elements 24 may be advanced through the sheath. Alternatively, while collapsed within the sheath, the electrode elements may be advanced to the thrombus together with the sheath.

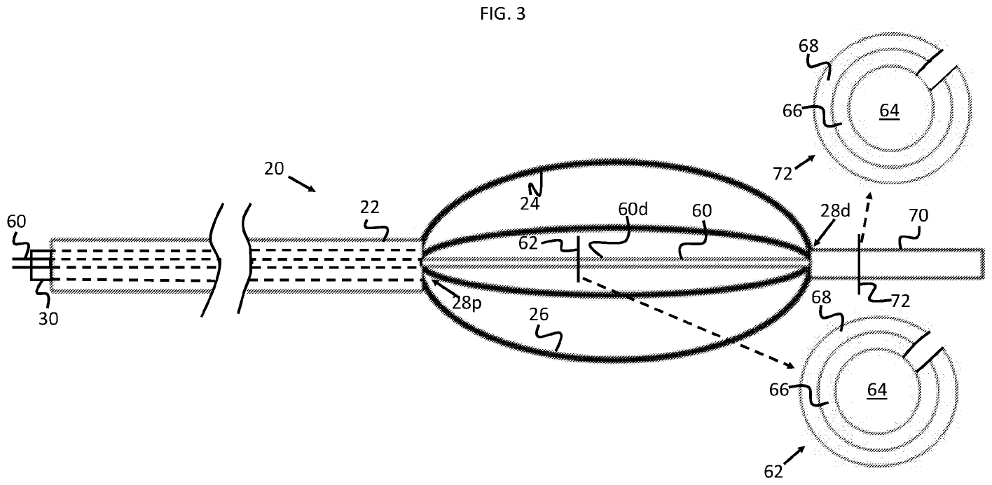

Electrode elements 24 are further configured to expand distally to the sheath following their advance to the thrombus. For example, the electrode elements may comprise a shape-memory material (e.g., nitinol) configured to expand so as define a predetermined shape upon exiting the sheath. Alternatively or additionally, as further described below with reference to FIG. 3, a pair of longitudinal elements coupled to the electrode elements may be used to expand the electrode elements. Typically, the electrode elements are lodged into the thrombus or moved into position alongside the thrombus (optionally such that the electrode elements contact the thrombus), prior to the expansion of the electrode elements.

In some embodiments, electrode elements 24 are configured to expand so as to define one or more circular or elliptical loops 26. For example, when expanded, electrode elements 24 may define an outer loop 26o and an inner loop 26i lying in the same plane. Alternatively, the electrode elements may define two loops lying in different respective planes, such as planes that are perpendicular to one another. Advantageously, loops 26 may entrap the thrombus.

Typically, the maximal width w0 of the widest loop 26 is greater than 2 mm and/or less than 30 mm, such as between 2 mm and 30 mm, e.g., 3-20 mm.

Typically, for embodiments in which apparatus 20 comprises multiple loops 26, the loops are coupled to one another (e.g., via any suitable adhesive) at a proximal junction 32p (located inside sheath 22 in FIG. 1) and a distal junction 32d.

In some embodiments, each loop 26 comprises a single electrode element (i.e., a single electrode element defines the loop). In other embodiments, at least one loop comprises multiple electrode elements, such as a pair of electrode elements. For example, outer loop 26o may comprise a first electrode element 24a and a second electrode element 24b, which are coupled to one another at proximal junction 32p and distal junction 32d.

Typically, when the electrode elements are expanded, the distance from the proximal end of the electrode elements to the distal end of the electrode elements (e.g., the distance from proximal junction 32p to distal junction 32d) is at least 10 mm and/or less than 100 mm, such as 10-100 mm, e.g., 20-80 mm.

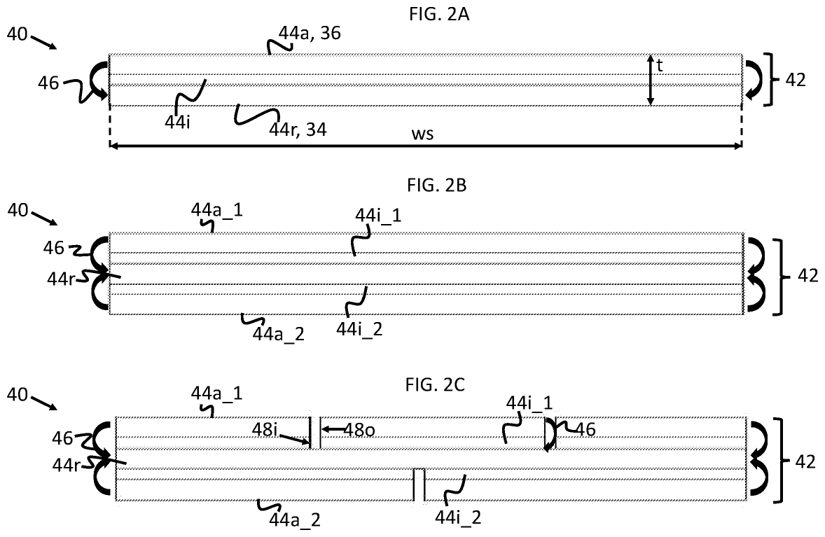

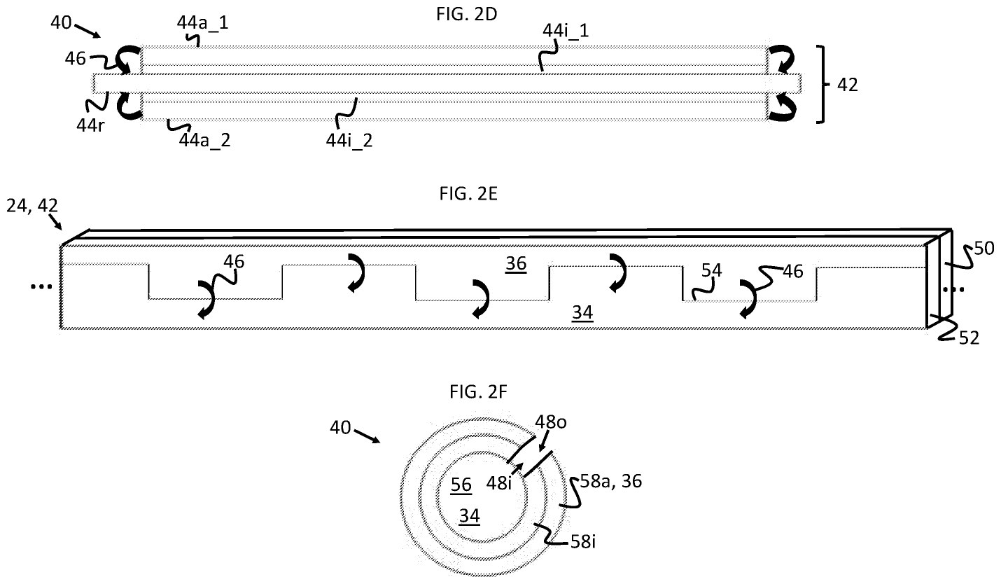

As further described below with reference to FIGS. 2A-F, each electrode element 24 comprises one or more reference electrodes 34 and one or more active electrodes 36. Active electrodes 36 are configured to attract the thrombus, following the expansion of the electrode elements, upon application of a voltage, by a power source 38, between active electrodes 36 and reference electrodes 34. (Typically, the voltage between the active electrodes and the reference electrodes is a positive voltage, such that the positively-charged active electrodes attract the negatively-charged thrombus.) Following the attachment of the thrombus to the active electrodes, apparatus 20, together with the thrombus, is removed from the body.

Typically, power source 38 is current-regulated, typically to 0.1-10 mA, e.g., 1-5 mA. In other embodiments, the power source is voltage-regulated, typically to 0.1-50 V, e.g., 1-40 V. The applied voltage may be constant or pulsed. Typically, the voltage is applied for a duration of between one second and 10 minutes, e.g., between five seconds and five minutes, such as between 10 seconds and two minutes.

Apparatus 20 further comprises one or more longitudinal elements coupled to the electrode elements and configured to pass through the sheath. Each of the longitudinal elements may facilitate control of the electrode elements and/or facilitate the attraction of the thrombus.

For example, apparatus 20 may comprise a proximally-coupled longitudinal element 30 (comprising, for example, a flexible hollow tube, a flexible solid wire, or a flexible solid shaft) coupled to the respective proximal ends of the electrode elements (e.g., to junction 32p). To advance the electrode elements from the sheath, the sheath may be withdrawn while a counterforce is applied to longitudinal element 30, or longitudinal element 30 may be pushed while a counterforce is applied to the sheath. (In each of the above cases, the counterforce may simply inhibit movement, or it may be sufficient to cause movement in the opposite direction.)

Alternatively or additionally, as shown in FIG. 3, apparatus 20 may comprise a distally-coupled longitudinal element 60 (comprising, for example, a flexible hollow tube, a flexible solid wire, or a flexible solid shaft) coupled to the respective distal ends of the electrode elements. Distally-coupled longitudinal element 60 may be used to advance the electrode elements from the sheath, as described above for proximally-coupled longitudinal element 30.

(In the context of the present application, including the claims, the “proximal end” of each electrode element is the proximal end of the electrode element when the electrode element is expanded. Similarly, the “distal end” of each electrode element is the distal end of the electrode element when the electrode element is expanded.)

Alternatively or additionally, apparatus 20 may comprise a first longitudinal wire (or “lead”) 28a, which is distally connected (e.g., soldered) to reference electrodes 34, and a second longitudinal wire (or “lead”) 28b, which is distally connected (e.g., soldered) to active electrodes 36. First wire 28a and second wire 28b are configured to connect to different respective terminals of power source 38, such that the power source may apply the voltage between the electrodes by applying the voltage between the first and second wires.

Alternatively, apparatus 20 may comprise multiple first wires 28a, each of which is connected to a different respective subset of the reference electrodes. (In such embodiments, one subset of reference electrodes may be activated by the power source without activating another subset of reference electrodes.) Alternatively or additionally, apparatus 20 may comprise multiple second wires 28b, each of which is connected to a different respective subset of the active electrodes. (In such embodiments, one subset of active electrodes may be activated by the power source without activating another subset of active electrodes.)

For embodiments in which proximally-coupled longitudinal element 30 or distally-coupled longitudinal element 60 is hollow, first wire 28a and/or second wire 28b may pass through the proximally-coupled or distally-coupled longitudinal element. Alternatively, first wire 28a and second wire 28b may run alongside the other longitudinal element(s).

In other embodiments, proximally-coupled longitudinal element 30 and/or distally-coupled longitudinal element 60 is connected to power source 38, and the voltage is applied to the electrodes via one or both of these longitudinal elements. For example, the proximally-coupled longitudinal element may connect one set of electrodes (e.g., the active electrodes) to one terminal of the power source, and the distally-coupled longitudinal element may connect the other set of electrodes (e.g., the reference electrodes) to the other terminal. Alternatively, either the proximally-coupled or distally-coupled longitudinal element may connect one set of electrodes to one terminal of the power source, and a wire may connect the other set of electrodes to the other terminal.

FIG. 1 marks a transverse cross-section 40 through an electrode element 24. Cross-section 40, per various different embodiments, is shown in FIGS. 2A-D, to which reference is now made.

In some embodiments, each of the electrode elements comprises a multilayered strip 42. The layers of strip 42 may be attached to each other using any suitable adhesive.

For example, a reference layer 44r of the strip may comprise reference electrodes 34, one or more active layers 44a of the strip may comprise active electrodes 36, and strip 42 may further comprise one or more insulating layers 44i that electrically insulate reference layer 44r from active layers 44a. Upon the application of the voltage, electric current 46 flows between the active layers and the reference layer.

Typically, the width ws of strip 42 (as indicated in FIG. 2A) is at least 0.1 mm and/or less than 2 mm, such as between 0.1 mm and 2 mm, e.g., 0.2-1 mm.

Typically, the thickness of each layer of strip 42 is less than 0.2 mm, such as less than 0.1 mm. Alternatively or additionally, the total thickness t of strip 42 (as indicated in FIG. 2A) may be less than 1 mm, such as less than 0.5 mm, regardless of the number of layers in the strip.

In FIG. 2A, active layers 44a consist of a single active layer, and insulating layers 44i consist of a single insulating layer disposed between the reference layer and the active layer. (Thus, strip 42 comprises three layers in total.) In some embodiments, the active layer faces inward (i.e., toward the longitudinal axis of apparatus 20), so as to better entrap the thrombus inside loops 26 (FIG. 1).

In FIG. 2B, active layers 44a comprise a first active layer 44a_1 and a second active layer 44a_2 disposed on opposite sides of reference layer 44r. Insulating layers 44_i comprise a first insulating layer 44i_1 disposed between first active layer 44a_1 and reference layer 44r, and a second insulating layer 44i_2 disposed between second active layer 44a_2 and reference layer 44r. (Thus, strip 42 comprises five layers in total.) An advantage of such embodiments is better entrapment of the thrombus due to the increased electric current 46 and the increased surface area of the active electrodes.

In FIG. 2C, each of the active layers is shaped to define one or more outer gaps 48o, and each of the insulating layers is shaped to define one or more inner gaps 48i at least partly aligned with outer gaps 480. An advantage of such embodiments is that additional current 46 may flow through the gaps. (For ease of illustration, this additional current is not shown in all of the gaps.) Similarly to the five-layered strip of FIG. 2B, the three-layered strip of FIG. 2A may be shaped to define outer gaps 48o and inner gaps 48i.

It is noted that each gap may have any suitable length. For example, a gap may extend throughout the length of the strip, such that the gap divides the layer of the strip into multiple disconnected segments.

In some embodiments, to facilitate the flow of additional current, at least one insulating layer 44i is narrower (e.g., 5-30% narrower) than reference layer 44r or the active layer 44a adjacent to the insulating layer. Both the three-layered strip of FIG. 2A and five-layered strip of FIG. 2B may comprise this feature. This feature may be combined with the gaps of FIG. 2C.

For example, in FIG. 2D, the insulating layers are narrower than reference layer 44r. Optionally, as shown in FIG. 2D, the active layers may have the same width as the insulating layers.

Alternatively, the insulating layers may be narrower than the active layers. Optionally, the reference layer may have the same width as the insulating layers.

Reference is now made to FIG. 2E, which is a schematic illustration of an electrode element 24 comprising a multilayered strip 42, in accordance with some embodiments of the present invention. As opposed to FIGS. 2A-D, which show a transverse cross-section through the electrode element, FIG. 2E shows the electrode element along its length.

In some embodiments, strip 42 comprises a substrate layer 50, which typically comprises a polymer such as polyimide. One or more electrode layers 52 are mounted to substrate layer 50, e.g., via any suitable adhesive. Each electrode layer 52 comprises a respective reference electrode 34 and a respective active electrode 36. For example, as shown in FIG. 2E, strip 42 may comprise a single electrode layer 52. Alternatively, strip 42 may comprise two electrode layers 52, each being mounted to a different respective side of substrate layer 50.

In such embodiments, each active electrode typically comprises one or more of the materials specified above, with reference to FIGS. 2A-D, for active layers 44a. Similarly, each reference electrode typically comprises one or more of the materials specified above for reference layers 44r.

In some embodiments, electrode layer 52 further comprises an electrically-insulating element 54 disposed between the active and reference electrodes. Typically, to facilitate the flow of current, electrically-insulating element 54 has a thickness that is less than 0.05 mm, such as less than 0.01 mm. In other embodiments, an air gap separates the two electrodes from one another.

Typically, to increase the length of the interface between the electrodes and thus increase the amount of current 46, the reference electrode and active electrode protrude into one another. For example, as shown in FIG. 2E, the electrodes may comprise interlocking square-wave edges. Alternatively, for example, the electrodes may comprise interlocking sinusoidal edges.

Reference is now made to FIG. 2F, which is a schematic illustration of another transverse cross-section 40 through an electrode element, in accordance with some embodiments of the present invention.

In some embodiments, each electrode element comprises a solid or hollow core 56, which comprises reference electrodes 34. For example, core 56 may comprise an electrically-conductive wire that functions as a reference electrode. Each electrode element further comprises an electrically-insulating layer 58i, which is wrapped around the core, and an active layer 58a, which comprises active electrodes 36 and is wrapped around electrically-insulating layer 58i.

Typically, in such embodiments, active layer 58a is shaped to define one or more outer gaps 48o, and electrically-insulating layer 58i is shaped to define one or more inner gaps 48i at least partly aligned with outer gaps 480. Thus, electric current may flow between the electrodes via the gaps.

For example, active layer 58a may comprise an electrically-conductive perforated tube that functions as active electrode 36, and electrically-insulating layer 58i may comprise another perforated tube having perforations at least partly aligned with those of active layer 58a. Alternatively, active layer 58a may comprise an electrically-conductive coil that functions as active electrode 36, and electrically-insulating layer 58i may comprise another coil, the windings of which are at least partly aligned with those of active layer 58a. Alternatively, either one of the layers may comprise a coil, and the other layer may comprise a perforated tube having perforations lying at least partly between the windings of the coil. As yet another option, active layer 58a may comprise a series of disconnected electrically-conductive tube segments that function as active electrode 36, and electrically-insulating layer 58i may comprise another series of disconnected tube segments at least partly aligned with those of active layer 58a.

Reference is now made to FIG. 3, which is a schematic illustration of apparatus 20, in accordance with some embodiments of the present invention. (For ease of illustration, first wire 28a and second wire 28b are omitted from FIG. 3.)

As described above with reference to FIG. 1, in some embodiments, apparatus 20 comprises a distally-coupled longitudinal element 60 coupled to the respective distal ends of the electrode elements (e.g., to distal junction 28d). For embodiments in which proximally-coupled longitudinal element 30 is hollow, distally-coupled longitudinal element 60 may pass through proximally-coupled longitudinal element 30.

Distally-coupled longitudinal element 60 may be used, together with proximally-coupled longitudinal element 30, to adjust the respective lengths and widths of loops 26. For example, a user grasping the proximal ends of distally-coupled longitudinal element 60 and proximally-coupled longitudinal element 30, which protrude from the proximal end of sheath 22, may slide the two longitudinal elements relative to one another. For example, to lengthen and narrow the loops, the user may push distally-coupled longitudinal element 60 while applying a counterforce to proximally-coupled longitudinal element 30. Conversely, to shorten and widen the loops, the user may push proximally-coupled longitudinal element 30 while applying a counterforce to distally-coupled longitudinal element 60. (In each of the above cases, the counterforce may simply inhibit movement, or it may be sufficient to cause movement in the opposite direction.)

Alternatively or additionally, regardless of whether apparatus 20 comprises loops 26, distally-coupled longitudinal element 60 may be used, together with proximally-coupled longitudinal element 30, to expand the electrode elements. For example, the user may push distally-coupled longitudinal element 60 while applying a counterforce to proximally-coupled longitudinal element 30.

Alternatively or additionally, regardless of whether apparatus 20 comprises loops 26, distally-coupled longitudinal element 60 may be used, together with proximally-coupled longitudinal element 30, to twist the electrode elements around the thrombus subsequently to the expansion of the electrode elements (and, typically, prior to the application of the voltage), so as to increase the contact between the electrode elements and the thrombus. In other words, proximally-coupled longitudinal element 30 may be rotated about its longitudinal axis while a counterforce is applied to distally-coupled longitudinal element 60, or distally-coupled longitudinal element 60 may be rotated while a counterforce is applied to proximally-coupled longitudinal element 30. (In each of the above cases, the counterforce may simply inhibit rotation, or it may be sufficient to cause rotation in the opposite direction.)

In some embodiments, distally-coupled longitudinal element 60—in particular, at least the distal portion 60d of distally-coupled longitudinal element 60, which is disposed between the proximal and distal ends of the loops when the loops are expanded—comprises a longitudinal-element active electrode, a longitudinal-element reference electrode disposed inside the longitudinal-element active electrode, and an electrically-insulating element disposed between the longitudinal-element reference electrode and the longitudinal-element active electrode. Thus, distally-coupled longitudinal element 60 may apply an additional attractive force to the thrombus. Typically, in such embodiments, distally-coupled longitudinal element 60 is shaped to define gaps passing through the longitudinal-element active electrode and the electrically-insulating element, so as to facilitate the flow of current between the longitudinal-element active electrode and the longitudinal-element reference electrode.

Alternatively or additionally to distally-coupled longitudinal element 60, apparatus 20 may comprise a distal electrode element 70 disposed at respective distal ends of the loops. Distal electrode element 70 comprises a distal active electrode, a distal reference electrode disposed inside the distal active electrode, and a distal electrically-insulating element disposed between the distal reference electrode and the distal active electrode. Thus, the distal electrode element may apply an additional attractive force to the thrombus. Typically, in such embodiments, the distal electrode element is shaped to define gaps passing through the distal active electrode and the electrically-insulating element, so as to facilitate the flow of current between the distal active electrode and the distal reference electrode.

Typically, the length of the distal electrode element is at least 10 mm and/or less than 100 mm, such as between 10 and 100 mm, e.g., 20-80 mm Typically, distal electrode element 70 is narrower than w0 (FIG. 1), so as to facilitate the removal of a thrombus from a distally-narrowing blood vessel (i.e., a blood vessel having a diameter that decreases in the distal direction), such as the pulmonary artery. In some embodiments, distal electrode element 70 is cylindrical, having an outer diameter that is typically between 0.5 mm and 4 mm, such as between 1 mm and 3 mm.

In some embodiments, as shown in FIG. 3, the distal electrode element is entirely distal to electrode elements 24. In other embodiments, the distal electrode element is only partly distal to electrode elements 24. For example, the proximal end of distal electrode element 70 may be disposed inside of loops 26.

In some embodiments, as shown in FIG. 3, the transverse cross-section 62 of distally-coupled longitudinal element 60, and/or the transverse cross-section 72 of distal electrode element 70, appears similar to the transverse cross-section 40 shown in FIG. 2F. For example, distally-coupled longitudinal element 60 and/or the distal electrode element may comprise: (i) an electrically-conductive core wire 64, which functions as a reference electrode, (ii) an electrically-insulating perforated tube 66 covering core wire 64, and (iii) an electrically-conductive perforated tube 68, which covers perforated tube 66, has perforations at least partly aligned with those of perforated tube 66, and functions as an active electrode. Alternatively or additionally, distally-coupled longitudinal element 60 and/or the distal electrode element may comprise any other suitable pair of gapped layers over core wire 64, the gaps in each layer being at least partly aligned with those in the other layer. The layers may comprise, for example, a pair of coils, a perforated tube and a coil, or two series of disconnected tube segments, as described above with reference to FIG. 2F.

In some embodiments, distally-coupled longitudinal element 60 (e.g., core wire 64 thereof) and/or distal electrode element 70 (e.g., core wire 64 thereof) is hollow. In such embodiments, a guidewire may be passed through distally-coupled longitudinal element 60 and/or through the distal electrode element.

In some embodiments, distal electrode element 70 is coupled to the distal ends of loops 26, e.g., to distal junction 28d. Alternatively or additionally, the distal electrode element may be coupled to distally-coupled longitudinal element 60. For example, a single core wire 64 may extend through both distally-coupled longitudinal element 60 and the distal electrode element. Alternatively, distally-coupled longitudinal element 60 may pass through the distal electrode element. (It follows, from the above, that distally-coupled longitudinal element 60 may be coupled to electrode elements 24 indirectly, via the distal electrode element.)

In some embodiments, distally-coupled longitudinal element 60 extends distally to the electrode elements. In such embodiments, the distal extension of distally-coupled longitudinal element 60 may have the features of distal electrode element 70 described above, such that apparatus 20 need not necessarily comprise distal electrode element 70.

In some embodiments, any reference electrodes belonging to distally-coupled longitudinal element 60 and distal electrode element 70 are connected to the same first wire 28a (FIG. 1) as are the reference electrodes belonging to electrode elements 24. Similarly, any active electrodes belonging to distally-coupled longitudinal element 60 and distal electrode element 70 are connected to the same second wire 28b (FIG. 1) as are the active electrodes belonging to electrode elements 24. In other embodiments, the active electrodes and/or reference electrodes belonging to distally-coupled longitudinal element 60 and/or distal electrode element 70 are connected, and thus activated, separately from the electrodes belonging to electrode elements 24.

In alternate embodiments, each loop comprises one or more active electrodes without necessarily comprising any reference electrodes, and distally-coupled longitudinal element 60 comprises one or more reference electrodes without necessarily comprising any active electrodes. Upon the application of a voltage between the active electrodes and the reference electrodes, electric current flows between the active electrodes and the references electrodes, and thus, the active electrodes attract the thrombus.

For example, each electrode element may comprise a wire that functions as an active electrode, without any additional layers. Similarly, distally-coupled longitudinal element 60 may comprise core wire 64, which functions as a reference electrode, without any additional layers.

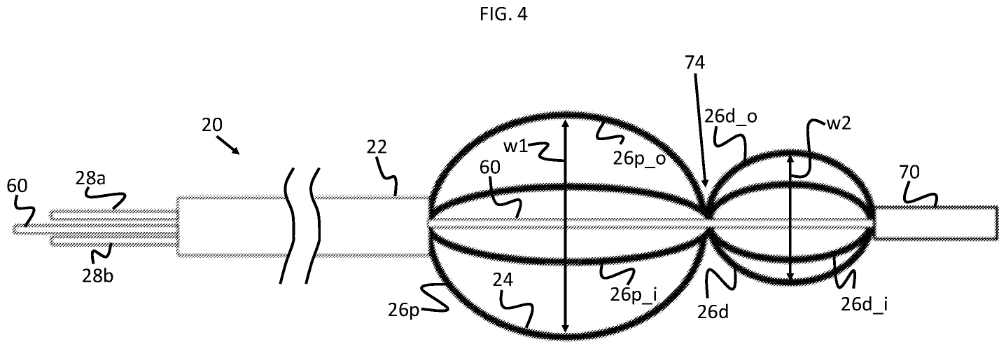

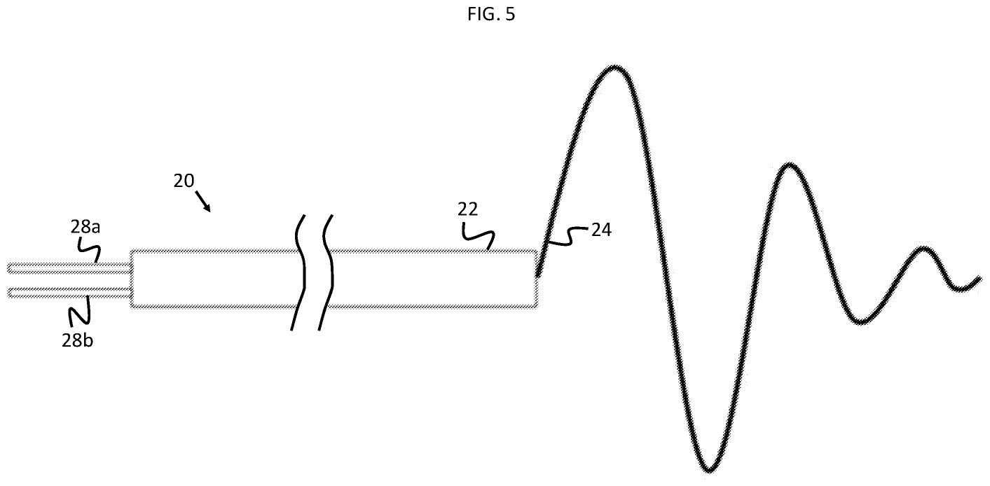

Reference is now made to FIG. 4, which is a schematic illustration of apparatus 20, in accordance with some embodiments of the present invention. (Proximally-coupled longitudinal element 30 is omitted from FIGS. 4-5 for ease of illustration.)

In some embodiments, electrode elements 24 define a proximal set of one or more proximal loops 26p and a distal set of one or more distal loops 26d. For example, proximal loops 26p may comprise an outer proximal loop 26p_o and an inner proximal loop 26p_i lying in the same plane, and distal loops 26d may similarly comprise an outer distal loop 26d_o and an inner distal loop 26d_i lying in the same plane as the proximal loops. Alternatively, the loops may have any other suitable configuration; for example, distal loops 26d may lie in a plane perpendicular to that in which proximal loops 26p lie.

Distal loops 26d are coupled to the respective distal ends of proximal loops 26p. For example, the proximal ends of the distal loops may be coupled to each other and to the distal ends of the proximal loops at an interloop junction 74.

Typically, the distal set has a maximal width w2 that is less than the maximal width w1 of the proximal set. (The maximal width of each set is the maximal width of the widest loop in the set. Typically, w1 is greater than 2 mm and/or less than 30 mm, such as between 2 mm and 30 mm, e.g., 3-20 mm) Advantageously, this feature facilitates the removal of a thrombus from a distally-narrowing blood vessel.

Apparatus 20 may comprise any number of additional sets of loops (typically having progressively smaller maximal widths) distal to distal loops 26d.

In some embodiments, apparatus 20 further comprises distally-coupled longitudinal element 60, which is coupled to the distal ends of the most distal loops, and/or distal electrode element 70, which is disposed at the distal ends of the most distal loops.

Electrode elements 24 may have any suitable multilayered configuration, such as any of the configurations described above with reference to FIGS. 2A-F. Alternatively, as described above with reference to FIG. 3, each electrode element may comprise an active electrode without any additional layers, and current may be passed between the electrode elements and distally-coupled longitudinal element 60.

Reference is now made to FIG. 5, which is a schematic illustration of apparatus 20, in accordance with some embodiments of the present invention.

In some embodiments, as shown in FIG. 5, at least one electrode element 24 has a sinusoidal shape when expanded. Alternatively or additionally, at least one electrode element may have a helical shape when expanded. Such electrode elements may have any suitable multilayered configuration, such as any of the configurations described above with reference to FIGS. 2A-F. Furthermore, such electrode elements may be combined with any of the features described above with reference to the previous figures, such as distally-coupled longitudinal element 60 (FIG. 3).

In general, to facilitate the removal of a thrombus from a distally-narrowing blood vessel, the electrode elements may be configured to expand to define any shape having a width that decreases moving distally along a distal portion of the shape, such as the distalmost 50%, 30%, or 10% of the shape. Examples of such shapes include a loop (as shown in FIG. 1, FIG. 3, and FIG. 4) and the sinusoidal shape of FIG. 5, the “width” of this latter shape being the width of the envelope of the sinusoid.

Typically, each of the active electrodes described herein comprises gold, platinum, and/or an alloy of platinum and iridium. Typically, each of the reference electrodes described herein comprises stainless steel, nitinol, and/or titanium. (As described above with reference to FIG. 1, a shape-memory material such as nitinol may facilitate the expansion of the electrode elements.) Typically, each of the insulating elements described herein comprises one or more biocompatible polymers such as polyether block amide, polyimide, or polyurethane.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

I claim:

1. Apparatus, comprising:
one or more longitudinal elements, configured to pass through a sheath within a body of a subject; and
one or more expandable multilayered electrode elements coupled to the longitudinal elements and configured to:
advance to a thrombus in the body while collapsed inside the sheath, and
expand distally to the sheath following the advance to the thrombus,
each of the electrode elements comprising:
one or more reference electrodes; and
one or more active electrodes configured to attract the thrombus, following the expansion of the electrode elements, upon application of a voltage between the active electrodes and the reference electrodes.

2. The apparatus according to claim 1, further comprising the sheath.

3. The apparatus according to claim 1, wherein the longitudinal elements comprise a proximally-coupled longitudinal element coupled to respective proximal ends of the electrode elements.

4. The apparatus according to claim 1, wherein the longitudinal elements comprise a distally-coupled longitudinal element coupled to respective distal ends of the electrode elements.

5. The apparatus according to claim 4, wherein the distally-coupled longitudinal element comprises:
a longitudinal-element active electrode;
a longitudinal-element reference electrode disposed inside the longitudinal-element active electrode; and
an electrically-insulating element disposed between the longitudinal-element reference electrode and the longitudinal-element active electrode.

6. The apparatus according to claim 5, wherein the distally-coupled longitudinal element extends distally to the electrode elements.

7. The apparatus according to claim 1, wherein the electrode elements are configured to expand so as to define one or more loops.

8. The apparatus according to claim 7, further comprising a distal electrode element disposed at respective distal ends of the loops and comprising:
a distal active electrode;
a distal reference electrode disposed inside the distal active electrode; and
a distal electrically insulating element disposed between the distal reference electrode and the distal active electrode.

9. The apparatus according to claim 7, wherein the loops include:
a proximal set of one or more proximal loops; and
a distal set of one or more distal loops coupled to respective distal ends of the proximal loops, the distal set having a maximal width that is less than that of the proximal set.

10. The apparatus according to claim 1, wherein at least one of the electrode elements has a sinusoidal shape when expanded.

11. The apparatus according to claim 1, wherein at least one of the electrode elements has a helical shape when expanded.

12. The apparatus according to claim 1, wherein the electrode elements are configured to expand to define a shape having a width that decreases moving distally along a distal portion of the shape.

13. The apparatus according to claim 1, wherein each of the electrode elements comprises a multilayered strip.

14. The apparatus according to claim 13,
wherein a reference layer of the strip comprises the reference electrodes,
wherein one or more active layers of the strip comprise the active electrodes, and
wherein the strip further comprises one or more insulating layers that electrically insulate the reference layer from the active layers.

15. The apparatus according to claim 14, wherein the active layers consist of a single active layer, and wherein the insulating layers consist of a single insulating layer disposed between the reference layer and the active layer.

16. The apparatus according to claim 14,
wherein the active layers comprise a first active layer and a second active layer disposed on opposite sides of the reference layer, and
wherein the insulating layers comprise:
a first insulating layer disposed between the first active layer and the reference layer; and
a second insulating layer disposed between the second active layer and the reference layer.

17. The apparatus according to claim 14, wherein each of the active layers is shaped to define one or more outer gaps, and wherein each of the insulating lavers is shaped to define one or more inner gaps aligned with the outer gaps.

18. The apparatus according to claim 14, wherein at least one of the insulating layers is narrower than (i) the reference layer, or (ii) an adjacent one of the active layers.

19. The apparatus according to claim 13, wherein the strip comprises:
a substrate layer; and
one or more electrode layers mounted to the substrate layer, each of the electrode layers comprising a respective one of the reference electrodes and a respective one of the active electrodes.

20. The apparatus according to claim 19, wherein the respective one of the reference electrodes and the respective one of the active electrodes protrude into one another.

21. The apparatus according to claim 1, wherein each of the electrode elements comprises:
a core, which comprises the reference electrodes;
an electrically-insulating layer, which is wrapped around the core; and
an active layer, which comprises the active electrodes and is wrapped around the electrically-insulating layer.

22. The apparatus according to claim 21, wherein the active layer is shaped to define one or more outer gaps, and wherein the electrically-insulating layer is shaped to define one or more inner gaps at least partly aligned with the outer gaps.

23. Apparatus, comprising:
one or more expandable electrode elements configured to advance to a thrombus in a body of a subject while collapsed inside a sheath within the body, and to expand distally to the sheath so as to define one or more loops following the advance to the thrombus; and
an electrically conductive longitudinal element coupled to respective distal ends of the electrode elements and configured to pass through the sheath,
each of the electrode elements comprising one or more active electrodes configured to attract the thrombus, following the expansion of the electrode elements, upon application of a voltage between the active electrodes and the longitudinal element.

24. A method, comprising:
inserting a sheath into a body of a subject;
advancing one or more expandable multilayered electrode elements to a thrombus in the body while the electrode elements are collapsed inside the sheath, each of the electrode elements including one or more reference electrodes and one or more active electrodes;
causing the electrode elements to expand distally to the sheath following the advance to the thrombus; and
causing the active electrodes to attract the thrombus, following the expansion of the electrode elements, by applying a voltage between the active electrodes and the reference electrodes.

25. The method according to claim 24, further comprising, following the expansion of the electrode elements, twisting the electrode elements around the thrombus by rotating one or more longitudinal elements that are coupled to the electrode elements and pass through the sheath.

26. The method according to claim 24,
wherein a longitudinal element is coupled to respective distal ends of the electrode elements and includes:
a longitudinal-element active electrode, and
a longitudinal-element reference electrode disposed inside the longitudinal-element active electrode, and
wherein the method further comprises applying the voltage between the longitudinal-element active electrode and the longitudinal-element reference electrode.

27. The method according to claim 24, wherein causing the electrode elements to expand comprises causing the electrode elements to expand so as to define one or more loops.

28. The method according to claim 27,
wherein a distal electrode element is disposed at respective distal ends of the loops and includes:
a distal active electrode, and
a distal reference electrode disposed inside the distal active electrode, and
wherein the method further comprises applying the voltage between the distal active electrode and the distal reference electrode.

29. The method according to claim 24, wherein each of the electrode elements includes a multilayered strip.

30. The method according to claim 29,
wherein a reference layer of the strip includes the reference electrodes,
wherein one or more active layers of the strip include the active electrodes, and
wherein the strip further includes one or more insulating layers that electrically insulate the reference layer from the active layers.

31. The method according to claim 29, wherein the strip includes:
a substrate layer, and
one or more electrode layers mounted to the substrate layer, each of the electrode layers including a respective one of the reference electrodes and a respective one of the active electrodes.

32. The method according to claim 24, wherein each of the electrode elements includes:
a core, which includes the reference electrodes,
an electrically-insulating layer, which is wrapped around the core, and
an active layer, which includes the active electrodes and is wrapped around the electrically-insulating layer.

33. A method, comprising:
inserting a sheath into a body of a subject;
advancing one or more expandable electrode elements to a thrombus in the body while the expandable electrode elements are collapsed inside the sheath,
each of the electrode elements including one or more active electrodes, and
an electrically-conductive longitudinal element being coupled to respective distal ends of the electrode elements;
causing the electrode elements to expand distally to the sheath so as to define one or more loops following the advance to the thrombus; and
causing the active electrodes to attract the thrombus, following the expansion of the electrode elements, by applying a voltage between the active electrodes and the longitudinal element.
